(19) United States
(12) Patent Application Publication
(10) Pub. No.: US 2025/0095859 A1
(43) Pub. Date: Mar. 20, 2025

(54) PATIENT-CENTRIC LOAD PLANNING SYSTEM AND METHOD

(71) Applicant: Siemens Healthcare Diagnostics Inc., Tarrytown, NY (US)

(72) Inventor: Yue Zhang, Jersey City, NJ (US)

(73) Assignee: Siemens Healthcare Diagnostics Inc., Tarrytown, NY (US)

(21) Appl. No.: 18/727,899

(22) PCT Filed: Jan. 24, 2023
(86) PCT No.: PCT/US2023/061131

§ 371 Date: Jul. 10, 2024

Related U.S. Application Data
(60) Provisional application No. 63/267,637, filed on Feb. 7, 2022.

(51) Int. Cl. G16H 50/30; G16H 10/40; G16H 10/60

(52) U.S. Cl. CPC G16H 50/30; G16H 10/40; G16H 10/60

(57) ABSTRACT

A method of patient-centric load planning for a diagnostic laboratory having a plurality of instruments includes receiving a list of patient samples, a list of requested tests to be performed for each of the patient samples, and patient-centric information for the patient providing each patient sample. The received data is used to determine a load plan for the instruments, the load plan including a menu of selected tests assigned to each instrument and an ordering of patient samples according to a priority score based on at least a portion of the electronic health record (EHR) of the patient providing the patient sample. A system for patient-centric load planning includes a plurality of instruments controlled by a system controller and computer server to formulate and execute the load plan.

This application claims benefit under 35 USC § 119(e) of U.S. Provisional Application No. 63/267,637, filed Feb. 7, 2022. The entire contents of the above-referenced patent application are hereby expressly incorporated herein by reference.

### BACKGROUND

Large scale diagnostic laboratories typically process thousands of biological samples a day and millions over the course of a year. This type of volume requires a significant number of instruments (e.g., 100 or more) as well as a robust connection between instruments with automation lines. The operation of such diagnostic laboratories requires consistent and continuous monitoring, evaluation and intervention by human operators to ensure that results are accurate and service level agreements are satisfied. A large-scale diagnostic laboratory also has opportunities in increasing efficiency that arise with the use of multiple instruments that have been assigned overlapping assay menus.

Existing test ordering workflow usually starts with a doctor visit. The doctor diagnoses the patients' health conditions and determines a list of relevant assays for testing. This list is then forwarded to laboratories together with one or more samples collected from the patient. In the lab processing step, all samples are treated with equal importance (except STAT samples) regardless the patients' health conditions. In other words, the lab binarizes the priorities of each sample to be either very urgent or not urgent at all. In a typical diagnostic laboratory, STAT samples will be processed in a short time frame, for example, two hours while all other samples will be processed in as many as three days or more. However, it is possible for customers with good conditions (low priorities) to place the same or similar test orders as customers with poor but not emergent conditions. Current lab processing workflow does not distinguish the two which potentially cause longer waiting time for the latter customer.

Faster and more accurate assignment of assays to instruments and a better ordering of samples can improve overall throughput and other operating metrics of the diagnostic laboratory.

### SUMMARY OF THE EMBODIMENTS

In a first aspect, a method of patient-centric load planning for a diagnostic laboratory including a plurality of instruments includes receiving, at a computer server, computer readable data including a list of patient samples; a list of requested tests to be performed for each of the patient samples; and patient-centric information for the patient providing each patient sample. The method further includes determining a load plan for the instruments from the computer readable data via a load planning module executing on the computer server, the load plan comprising computer executable instruction configured to assign a menu of selected tests to each instrument; order the patient samples according to a priority score based on at least a portion of the electronic health record (EHR) of the patient providing the patient sample; and process the patient samples using instruments assigned to perform the requested tests for each patient sample.

In a second aspect, a system for patient-centric load planning for a diagnostic laboratory includes a system controller, a plurality of instruments controlled by the system controller to perform one or more requested tests on one or more patient samples, and a computer server coupled to the system controller, the computer server comprising a load planning module for determining a load plan for the plurality of instruments.

In a third aspect, a non-transitory computer readable storage medium includes a load planning module for performing patient-centric load planning for a diagnostic laboratory.

### BRIEF DESCRIPTION OF THE FIGURES

FIG. 1 is a flowchart of a method of patient-centric load planning for a diagnostic laboratory, in an embodiment.

FIG. 2 is a more detailed flowchart of a step of the method of FIG. 1, in embodiments.

FIG. 3 is a more detailed flowchart of another step of the method of FIG. 1, in embodiments.

FIG. 4 is a schematic block diagram of a system for performing the method of FIG. 1, in an embodiment.

### DETAILED DESCRIPTION OF THE EMBODIMENTS

Opportunities to increase efficiency in a diagnostic laboratory increase in relationship to the number of instruments used in the laboratory, particularly when multiple instruments have overlapping test (e.g., assay and/or clinical chemistry) menus. As used herein, instruments may also be referred to as laboratory analyzers and/or machines (such as immuno-assay instruments, clinical chemistry analyzers, and in vitro analyzers). Creating an optimal assignment of requested tests (e.g., assays and/or clinical chemistry) across the instruments can consider multiple factors, including patient-centric information.

Patient-centric lab load planning can help provide more insight into the order of processing samples beyond the binary designation of STAT or non-STAT assigned by the doctor ordering the test(s). It can also help determine an optimal assay menu for each instrument in the diagnostic laboratory and forecast future demand for reagent quantity by predicting the tests that will be ordered by the same customer in the future orders.

As described herein, patient urgency/priority is encoded in a continuous scalar, named the priority score, which is developed from the patient's Electronic Health Record (EHR). Jointly with other lab processing objectives and constraints as described in PCT/US2020/035507, incorporated by reference, such as load balancing and quality control cost, the priority score is used to find an optimal reagent load plan to minimize turnaround time for sample orders with high priorities. Further, the priority encoding system also produces patients' unique features, namely the finger-print features, which are used for future reagent demand planning.

Instead of treating patient priority as a binary class (STAT vs non-STAT), embodiments disclosed herein encode patient priority as a continuous scalar. Further, this priority encoding is then used for load plan optimization, where the related objective is to minimize number of sample stops for a patient with high priority.

Optimization systems and methods according to embodiments include configurable constraints and objective functions that can be tailored to the unique needs of a laboratory. Objective functions associated with patient-centric information are disclosed herein, but other objective functions e.g., quality assurance cost, test assignment redundancy, workload balancing across laboratory analyzers, may be combined in an overall load plan for a diagnostic laboratory in some embodiments. In addition to the constraints discussed herein, other constraints may include, e.g., the number of available reagent pack slots or wedges in a reagent carousel of an instrument; initial reagent pack volumes; and configured test menus.

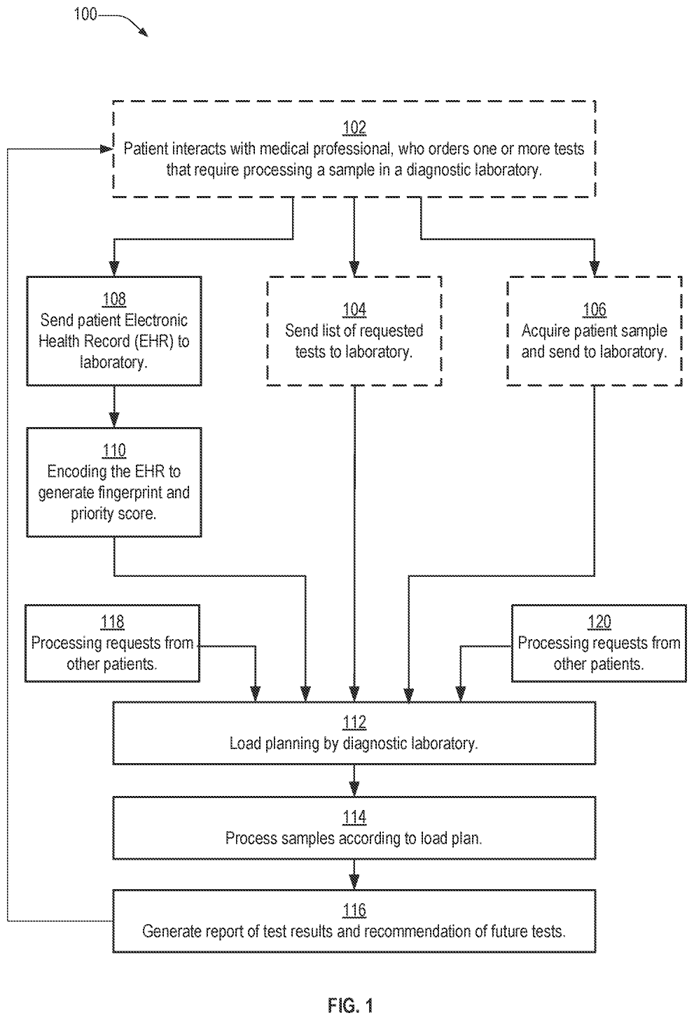

FIG. 1 is a flowchart of a method 100 of patient-centric load planning for a diagnostic laboratory, in an embodiment. Method 100 includes steps 108, 110, 112, 114, 116, 118 and 120. In embodiments, method 100 also includes at least one of steps 102, 104 and 106.

Step 102 includes a patient interaction with a medical professional. In an example of step 102, a patient interaction may be in person or virtual, and the medical professional is a doctor or any medical professional who is able to order tests, or assays, on a biological sample given by the patient. Discussion herein referring to a doctor should be understood as applying to any medical professional with the ability to order tests performed.

Step 104 includes sending a list or requested tests to a laboratory. Step 106 includes acquiring a patient sample and sending it to the laboratory. In an example of steps 104 and 106, the list of test requests may be sent electronically, and the patient sample may be acquired during the visit to the doctor or during a separate appointment with a different provider. Other mechanisms for delivering test requests and samples may be used.

Step 108 includes sending all of or a portion of the patient's Electronic Health Record (EHR) to the diagnostic laboratory. In an example of step 108, an EHR includes text and images that have been generated through appointments with doctors and other medical professionals to describe a patient's medical history.

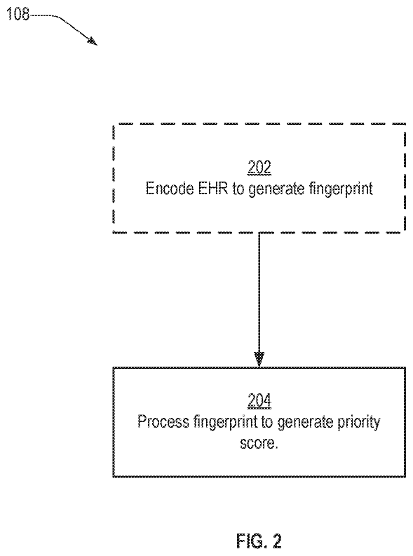

Step 110 includes encoding the EHR to generate a fingerprint and priority score, then sending them to the diagnostic laboratory. Step 112 includes processing the fingerprint, priority score and list of requested tests to generate a load plan for the diagnostic laboratory. In an example of step 112, a load plan is developed based on a group of samples. In addition to the sample and test requests from patient interaction 102, any number of sample processing requests may be received by a diagnostic laboratory, represented at 118 and 120. A load plan includes assigning a menu of selected tests to each instrument in the diagnostic laboratory and ordering the patient samples according to the priority score of the patient providing the patient sample. Steps 110 and 112 will be described in more detail in connection with FIG. 2.

Step 114 includes processing samples by according to the load plan. Samples may be grouped to develop a load plan based on a number of criteria, including a period of time during which the samples are received or a source of the samples, for example. In addition to the patient-centric load planning described above, a load plan may also include other criteria for optimizing operational efficiency, such as:
1) Reduced Turn-Around-Time (TAT): Reducing TAT is crucial for increasing throughput, staying within the limits of service level agreements, and responding to time sensitive requests.
2) Load Balancing: A balanced load across each instrument can improve overall TAT and prevent excessive wear-out.
3) Efficient Reagent Usage: Reagent is a large cost associated with diagnostic test operations. Optimizing reagent use is a priority in operational considerations.
4) Quality Assurance Costs: Each instrument goes through quality control and calibration steps incurring a cost that has to be managed for optimal efficiency.
5) Improved System Robustness: Ensuring robustness of the whole laboratory setup to cover all the tests against instrument failures is necessary.

Step 116 includes generating a report of test results and recommendation future test results. In an example of step 116, the patient fingerprint generated in step 110 allows the diagnostic laboratory to forecast future test requests that may be associated with this patient. This also allows the laboratory to forecast future requirements for reagents or other resources used by the laboratory. The fingerprint may be used to access a database of stored fingerprints and associated tests or processed using a neural network, for example. Other methods of correlating a patient fingerprint to similar fingerprints from other patients may be used to make predictions. A report of the test results and a recommendation of possible future tests to be requested is generated and sent back to the requesting doctor as explained in more detail below.

FIG. 2 is a more detailed flowchart of step 108 of method 100.

Patient-centric load planning uses a patient's Electronic Health Record to provide information relevant to sample processing priority and test menu assignments to instruments in a diagnostic laboratory.

In step 202, an encoder-decoder structured network may be used for EHR matrix reconstruction and fingerprint generation. In an embodiment, an EHR is formulated as a matrix and processed using a representation learning model that transforms patient EHR matrixes into low-dimensional, dense vectors. These vectors may be understood as including a number of features and represent a fingerprint of the patient's EHR. Processing an EHR matrix to generate a fingerprint may use a combination of embedding, convolutional neural networks (CNNs) and/or autoencoders (AEs) to derive vector-based patient representations that can be used for clinical inference and medical analysis. In model training, the data used to train the system will be sample EHRs with a binary STAT label.

In step 204, the fingerprint feature is further processed to generate a priority score. In an embodiment, the vector-based fingerprint is processed to predict whether the patients' sample is urgent (STAT) or not. A multi-layer perceptron (MLP) may be used to convert the fingerprint to a scalar priority score, having a value between 0 and 1. The priority score indicates how likely this patient sample is an urgent one. This priority score and finger-print feature will be transferred together with the sample and order of test requests from the doctor to the lab.

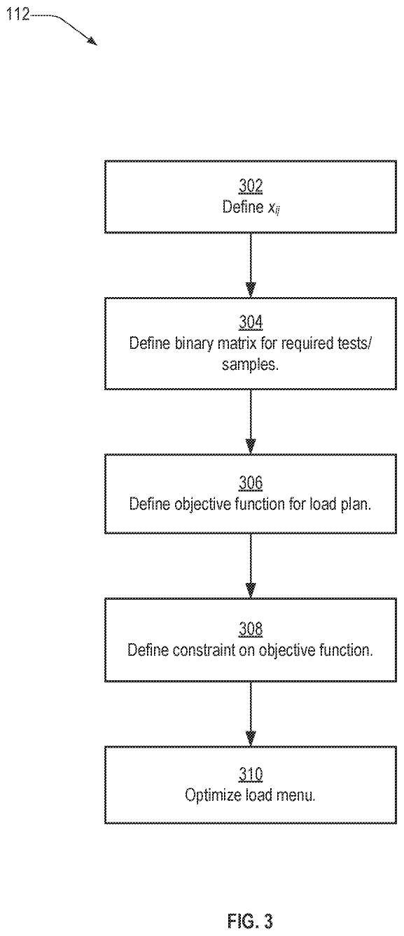

FIG. 3 is a more detailed flowchart of step 112 of method 100. A large-scale diagnostic laboratory may include a significant number of instruments as well as a robust connection between instruments with automation lines. An instrument may have the capability to perform several different tests, or assays, but is typically set up to turn on a selected subset, or menu, of tests at a given time. The set-up process includes loading the instrument with the reagent necessary to perform the menu of tests selected for the instrument. Given a cluster of samples, a load plan is developed to determine which instrument to load with which menu of tests, such that it is possible for sample orders with high priorities to make as few stops at different instruments as possible. A cluster of samples may be based on criteria including a period of time during which the samples are received or a source of the samples, for example.

A trivial solution for load planning is to deploy a full test menu to each instrument. In this solution, any sample will only make one stop and thus, a low turnaround time, depending on how many tests are required and if any of them are from different assay families, and also given enough reagent is load is loaded onto an instrument. However, this solution results in extra cost in quality control and calibration, as well as potential reagent waste for the lab. In another solution, when instrument menus are given, the problem of minimizing number of sample stops can be treated as a set-cover problem. Given a sample s that orders n different assays t, given by s={t₁, t₂, . . . , tₙ}, and existing menus x that are turned on in each machine, for example, x₁={t₁, t₃}, x₂={t₂, t₃, t₅, . . . }, . . . , xₘ={t₂, tₙ}, the set cover problem tends to find a minimal collection of x, such that s∈∪x.

Patient-centric load balancing can optimally determine test menus and reagent loadings with an objective of optimizing sample stops. In the method of FIG. 3, a patient priority score w is incorporated for a weighted set cover approach with a unified priority weight scalar wᵢ for the ith sample/patient, where the weight of a cover for one single sample is defined as wᵢ*|∪x|, i.e., the sample weight it covers multiplied by the total number of sets in the cover. In an embodiment, a load plan includes a list of x, i.e. menu configurations for all of the instruments, such that:
1) each sample should be covered by a collection of x, and
2) the total weight of all covers is minimized. For samples with larger weights (a higher priority score), a cover with high cardinality will result in a large product of wᵢ*|∪x|. Thus, the optimization algorithm will have a special focus on minimizing cover size of highly weighted samples.

In step 302, a variable xᵢⱼ is defined. In an example of step 302, xᵢⱼ is a binary payload variable which indicates the current distribution of assays, j∈ [symbol] , across instruments, i∈ [symbol] :

$${x}_{ij} = \{ \begin{matrix}1 , & if instrument i is configured to run test j , \\ 0 , & otherwise .\end{matrix}$$

In step 304 define S∈ [symbol] In an example of step 304, [symbol] is the set of all patient samples, [symbol] is the set of all assays and S is a binary matrix encoding the required tests for each sample such that:

$${s}_{aj} = \{ \begin{matrix}1 , & if sample a requires test j \\ 0 , & otherwise .\end{matrix}$$

Where a∈ [symbol] and [symbol] .

In step 306, define an objective function for the load plan. In an example of step 306, the objective function is defined as the total number of sample stops, weighted by each sample's priority:

$${𝒞}_{stops} = {w}_{a} {I}_{ai}$$

where Iₐᵢ is a binary variable indicating whether sample a will require instrument i:

$${I}_{ai} = \{ \begin{matrix}1 , & if sample a requires instrument i , \\ 0 , & otherwise .\end{matrix}$$

Minimizing [symbol] ₛₜₒₚₛ will encourage sample a to request as few instruments as possible, [symbol] u especially for highly weighted samples. Thus, it reduces the number of stops it makes, directly optimizing turn-around time.

In step 308, a constraint on the objective function is defined. In an example of step 308, in minimizing the number of total stops made by all the samples as given in the objective above, there is also a constraint that there is at least one set (or subset) of instruments to perform the tests required. The set of tests requested by sample a is denoted by

$${ℛ}_{a} = \{ j \vert {S}_{aj} > 0 , j ∈ 𝒯 \}$$

and the set of instruments that have been assigned to perform test j is denoted by

$${𝒦}_{j} = \{ i \vert {x}_{ij} = 1 , i ∈ , for a given test j \} .$$

Then the constraint that each sample must be covered can be written as follows:

$${\sum }_{i ∈ {𝒦}_{j}} {I}_{ai} \geq 1 , ∀ a ∈ , j ∈ {ℛ}_{a} .$$

It is important to note that the term is [symbol] Iₐᵢ is a non-linear combination (multiplication) of two indicator variables, xᵢⱼ and Iₐᵢ, that are both tied to the optimization variables nᵢⱼ. This non-linearity imposes additional computational complexity and to alleviate this we relax this combination by introducing an additional binary slack variable IIₐᵢⱼ and transform the original constraint into following:

$${\sum }_{i ∈ ℳ} {II}_{aij} \geq 1 , ∀ a ∈ , j ∈ {ℛ}_{a}$$

with IIₐᵢⱼ<xᵢⱼ, # a cannot stop at i for any j if i does not turn on j
   Iₐᵢⱼ≤Iₐᵢ, # a cannot do j at i if a does not stop at i
   IIₐᵢⱼ≥xᵢⱼ+Iₐᵢ−1, ∀i∈ [symbol] , j∈ [symbol] .# equivalent to if xᵢⱼ=Iₐᵢ==1, then IIₐᵢⱼ=1.

Step 310 includes optimizing the load plan. In an example of step 310, patient-centric load planning is given by the objective and the constraint of:

$$Minimize f ( x ) + {𝒞}_{stops}$$

$$Subject to g ( x ) \geq 0 , h ( x ) \geq 0 .$$

Where f(x) and g(x) refer to the typical objectives and constraints in load planning optimization such as machine load balancing, machine runtime, quality assurance cost and lab capacity as described above, h(x) is the sample stop constraints introduced above. Solving this optimization will return an optimized menu where the total number of weighted sample stops will be optimized for patients with different priorities.

Another part of the load planning is to determine the future reagent demand. In embodiments, a database-based approach may be used to predict future orders. A database may include, for example, the following three key entities: {f,S^{t}}, where f represents fingerprint features collected when training the encoder in step 202 of FIG. 2, S is the sample order matrix and t is the time of ordering. This database is built with a large amount of historical orderings from patient data.

When receiving the new test orders, the diagnostic laboratory uses the fingerprint features to query the database to find the closest triplet, denoted as {fᵢ, Sᵢ^{t},}. Then, the sample orders of Sᵢ^{t+1} may be used as the predicted ordering for this patient. In contrast to methods that predict orderings using only the test information, this additional query process finds those patients who have the most similar health conditions with the current patient providing a sample by using the fingerprint features, and then uses the future orderings of similar patients as a prediction for future orderings for the current patient. Over time, gathering all the predictions for new orders provides a predicted test demand for the entire laboratory. In embodiments, the predicted test ordering of Sᵢ^{t+1} may be provided as a recommendation for follow-up tests together with the actual test results in a report to be sent back to the doctor and patients, as shown in step 116 of FIG. 1.

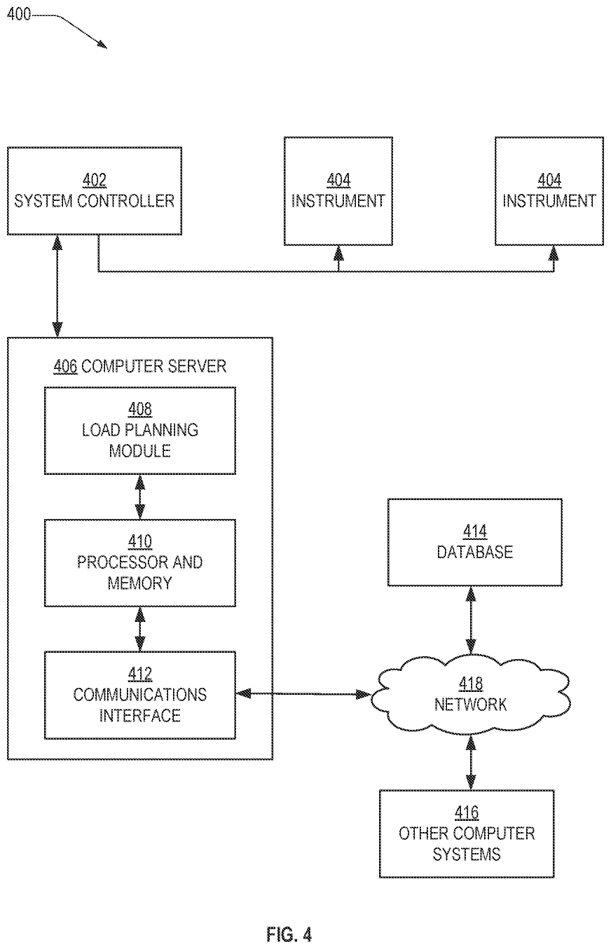

FIG. 4 is a schematic block diagram of a system 400 for performing the method of FIG. 1, in an embodiment. System 400 includes a plurality of instruments 404 for processing samples according to a menu of tests. One or more system controllers 402 may be provided for controlling instruments 404. and any automated systems for transferring samples between instruments. System 400 may include other components, equipment, and devices (not shown), such as, e.g., various sensors, barcode readers, robotic mechanisms, sample container loading areas, pre-processing stations which are used in performing typical sample processing tasks in a large-scale diagnostic laboratory. Instruments 404 are configured to perform one or more types of diagnostic tests and/or analyses on biological samples. In some embodiments, system 400 may include 100 or more instruments 404, each representing any inventory-consuming diagnostic capability, such as, e.g., chemistry, immune-assay, hematology, or molecular. In some embodiments, many instruments 404 may be capable of performing the same menu of tests, while other instruments 404 may be capable of performing only a limited number of tests or only certain individual tests.

System controller 402 is communicatively coupled to computer server 406. Computer server 406, which in some embodiments may be cloud based, may be any suitable computer device, and includes, for example a processor and memory 410 configured to store programming instructions and other information/data for execution by the processor. Computer server 406 may further include a communication interface 412 via which computer server 406 may be coupled to and in electronic communication with system controller 102 and network 418. Network 418 may include the Internet, a local area network (LAN) or a wireless local area network (WLAN), for example.

Computer server 406 may also include a load planning module 408. Load planning module 408 may be stored and executed by processor and memory 410. In alternative embodiments, the optimization-based load planning module 408 may be stored in other non-transitory computer readable storage mediums or accessed over network 418. Load planning module 408 includes computer executable instructions and may be configured and operable to receive and process input data to create a load plan. Input data includes computer readable data representing at least an inventory of the plurality of instruments 404 included in system 400, types and numbers of requested tests to be performed by system 400 that are received from other computer systems 416, such as computer systems associated with doctors or other medical professionals. Input data may also be received through direct input in the diagnostic laboratory or through other means.

A load plan created by load planning module 408 may include computer executable instructions configured to cause system controller 402 to schedule and direct each of the requested tests to be performed at one or more selected instruments 404 of system 400 in accordance with the objectives (preferences/priorities) received in the input data. A load plan as disclosed herein may indicate for each selected instrument 404, a selected one or more types of tests to be performed thereat, a number of requested tests to be performed thereat, and an order in which the number of requested tests are to be performed.

Computer server 406 may also access one or more databases over network 418 as shown in FIG. 4 or directly through communications interface 412.

System 400 may include other components which are not shown in FIG. 4 such as user interfaces or output devices, for example.

Changes may be made in the above methods and systems without departing from the scope hereof. It should thus be noted that the matter contained in the above description or shown in the accompanying drawings should be interpreted as illustrative and not in a limiting sense. Herein, and unless otherwise indicated: (a) the adjective “exemplary” means serving as an example, instance, or illustration, and (b) the phrase “in embodiments” is equivalent to the phrase “in certain embodiments,” and does not refer to all embodiments. The following claims are intended to cover all generic and specific features described herein, as well as all statements of the scope of the present method and system, which, as a matter of language, might be said to fall therebetween.

What is claimed is:

1. A method of patient-centric load planning for a diagnostic laboratory including a plurality of instruments, comprising:
receiving, at a computer server, computer readable data comprising:
a list of patient samples;
a list of requested tests to be performed for each of the patient samples; and
patient-centric information for the patient providing each patient sample, and
determining a load plan for the instruments from the computer readable data via a load planning module executing on the computer server, the load plan comprising computer executable instruction configured to:
assign a menu of selected tests to each instrument;
order the patient samples according to a priority score based on at least a portion of the electronic health record (EHR) of the patient providing the patient sample; and
process the patient samples using instruments assigned to perform the requested tests for each patient sample.

2. The method of claim 1, wherein determining a load plan further comprises minimizing the number of instruments that are needed to perform the requested tests for a patient sample.

3. The method of claim 2, wherein minimizing the number of instruments is performed for all patient samples in the list of patient samples according to the priority score for each patient sample.

4. The method of claim 1, further comprising encoding at least a portion of the patient's EHR into a vector-based fingerprint representing the patient's health.

5. The method of claim 4, further comprising processing the vector-based fingerprint using a multi-layer perceptron (MLP) to output the priority score.

6. The method of claim 4, further comprising:
accessing a database using the vector-based fingerprint to determine a predicted set of tests for the patient;
providing results of the requested tests and the predicted set of tests to a medical professional; and
predicting a future test demand for the diagnostic laboratory using the predicted set of tests.

7. The method of claim 1, further comprising determining a load plan optimized for other priorities including at least one of reduced turn-around-time (TAT), load balancing, efficient reagent usage, lower quality assurance costs, and improved system robustness.

8. The method of claim 1, wherein a menu of selected tests further comprises a set of tests that are enabled on an instrument and provided with a quantity of reagent for performing each test of the set of tests.

9. The method of claim 8, the load plan comprising computer executable instructions indicating an assignment of all requested tests to at least some instruments such that each instrument has sufficient reagent to perform all requested tests assigned thereto.

10. A system for patient-centric load planning for a diagnostic laboratory, comprising:
a system controller;
a plurality of instruments controlled by the system controller to perform one or more requested tests on one or more patient samples; and
a computer server coupled to the system controller, the computer server comprising a load planning module for determining a load plan for the plurality of instruments, the load plan comprising computer executable instruction configured to:
assign a menu of selected tests to each of the plurality of instruments;
order the patient samples according to a priority score based on at least a portion of an electronic health record (EHR) of the patient providing the patient sample; and
process the patient samples using instruments assigned to perform the requested tests for each patient sample.

11. The system of claim 10, wherein determining a load plan further comprises minimizing the number of instruments that are needed to perform the requested tests for a patient sample.

12. The system of claim 11, wherein minimizing the number of instruments is performed for all patient samples in the list of patient samples according to the priority score for each patient sample.

13. The system of claim 10, further comprising encoding at least a portion of the patient's EHR into a vector-based fingerprint representing the patient's health.

14. The system of claim 13, further comprising processing the vector-based fingerprint using a multi-layer perceptron (MLP) to output the priority score.

15. The system of claim 13, further comprising:
accessing a database using the vector-based fingerprint to determine a predicted set of tests for the patient;
providing results of the requested tests and the predicted set of tests to a medical professional; and
predicting a future test demand for the diagnostic laboratory using the predicted set of tests.

16. A non-transitory computer readable storage medium comprising a load planning module having computer executable instructions configured to cause a computer server to perform patient-centric load planning for a diagnostic laboratory to:
determine a load plan for the plurality of instruments from the computer readable data, the load plan comprising computer executable instruction configured to:
assign a menu of selected tests to each instrument;
order the patient samples according to a priority score based on at least a portion of an electronic health record (EHR) of the patient providing the patient sample; and
process the patient samples using instruments assigned to perform the requested tests for each patient sample.

17. The non-transitory computer readable storage medium of claim 16, wherein determining a load plan further comprises minimizing the number of instruments that are needed to perform the requested tests for a patient sample.

18. The non-transitory computer readable storage medium of claim 16, wherein minimizing the number of instruments is performed for all patient samples in the list of patient samples according to the priority score for each patient sample.

19. The non-transitory computer readable storage medium of claim 16, further comprising encoding at least a portion of the patient's EHR into a vector-based fingerprint representing the patient's health.

20. The system of claim 19, further comprising:
accessing a database using the vector-based fingerprint to determine a predicted set of tests for the patient;
providing results of the requested tests and the predicted set of tests to a medical professional; and
predicting a future test demand for the diagnostic laboratory using the predicted set of tests.
